(19) United States
(12) Patent Application Publication
(10) Pub. No.: US 2023/0043267 A1
(43) Pub. Date: Feb. 9, 2023

(54) SYSTEM AND METHOD FOR INFRARED DEHYDROFREEZING AND DEHYDRO FREEZE-DRYING

(71) Applicants: TIMOTHY CHILDS, SAN FRANCISCO, CA (US); SCOTT ROGER FORSBERG, MORGAN, UT (US)

(72) Inventors: TIMOTHY CHILDS, SAN FRANCISCO, CA (US); SCOTT ROGER FORSBERG, MORGAN, UT (US)

(21) Appl. No.: 17/877,858

(22) Filed: Jul. 29, 2022

Related U.S. Application Data
(63) Continuation of application No. 16/879,715, filed on May 20, 2020, now abandoned.
Application No. 16/879,715 is a continuation of application No. PCT/US19/53687, filed on Sep. 27, 2019.
(60) Provisional application No. 62/850,648, filed on May 21, 2019.

(51) Int. Cl. A23L 3/44; A61L 2/00; A01N 3/00; A23L 3/005; A23B 7/01; A23B 7/06; A23B 9/04; A23B 9/08; A23B 7/024

(52) U.S. Cl. CPC A23L 3/44; A61L 2/0011; A01N 3/00; A23L 3/0055; A23B 7/01; A23B 7/06; A23B 9/04; A23B 9/08; A23B 7/024; A23V 2002/00; A61L 2202/21

(57) ABSTRACT

Dehydration and drying are some of the oldest methods of preserving food and other biological materials such as proteins and both live and dead live microorganisms or their metabolites. The invention disclosed herein seeks a method of further improving the process of preserving biological material by combining the freezing or freeze-drying process with a partial infrared dehydration step. By using infrared radiation energy to perform simultaneous blanching and dehydration, the drying speed is greatly increased and better cellular and molecular integrity is maintained. The invention herein is also particularly useful in connection with cannabis and hemp.

### PRIORITY CLAIMS

This application is a continuation of U.S. patent application Ser. No. 16/879,715, filed May 20, 2020, which claims the priority benefit of U.S. Provisional Patent Application Ser. No. 62/850,648, filed on May 21, 2019, and PCT Application Serial Number PCT/US19/53687, filed on Sep. 27, 2019. Each of the foregoing applications are hereby incorporated by reference.

### BACKGROUND OF THE INVENTION

Dehydration and drying are some of the oldest methods of preserving food and other biological materials such as proteins and both live and dead live microorganisms or their metabolites. Since drying reduces the moisture in these materials making them lightweight and convenient to store, it can easily be used in place of other preservation techniques. In fact, one can even use drying along with other preservation techniques such as freezing or canning, which would make the process of preservation even better.

Drying food is simple, safe and easy to learn. The early American settlers practiced drying food using the natural forces of sun and wind and today, the use of technology has revolutionized this method of preserving food. With modern food dehydrators, foods such as fruit leathers, fruit chips, dried nuts and seeds and meat jerky, can all be dried year-round at home.

Being easy to store and carry and requiring no refrigeration makes dried foods ideal for domestic use as well as for use in the rough outdoors or other applications, such as military operations, disaster relief and space travel.

Moreover, dried foods are good sources of quick energy and wholesome nutrition, since the only thing lost during preservation is moisture. For instance, meat jerky, dried nuts and seeds are good sources of protein for a snack or a meal. The fruit leathers and chips provide plenty of quick energy. Dried vegetables, too, can be used to prepare wholesome casseroles and soups and the nutritional value can be enhanced by using the soaking water for cooking. Therefore, dried foods are an easy food option for busy executives, hungry backpackers and active women and children, soldiers and astronauts, all of whom can benefit from the ease of use and nutritional content of dried foods.

Fruits, vegetables, nuts and grains, those items are commonly dried using a process known as steam blanching. Blanching is the process whereby foods are briefly cooked in boiling water, steam, or syrup such that it destroys enzymes that catalyze the reaction of food spoilage. This type of drying also serves to weaken fibers on the surface of fruits or vegetables, allowing dehydration and rehydration to occur more efficiently.

Another method of blanching food for preservation is water blanching. Using this technique, the food is scalded in boiling water and then removed after a brief interval, and finally plunged into ice water or placed under cold running water to halt the cooking process.

While each of these methods is commonly used to produce preserved food, they each have their drawbacks which relate to the amount of nutrients, texture, and color that is lost in the process. is especially important when considering the preservation of living systems and molecules or metabolites created by those systems such as in probiotics, vaccines and other products derived from microbes, microbial fermentation or synthetic biology. Insufficient drying can lead to excess water activity which has a deleterious effect on shelf life by preventing organisms from achieving stasis. Drying too slowly or allowing the temperature to hover or fluctuate near the freezing point of water during the drying process can lead to the formation of ice crystals which can damage cell walls and membranes. In the case of metabolites or products of synthetic biology, poorly differentiated heat energy can alter molecular integrity, especially for proteins, enzymes and pharmaceuticals which depend on exacting stereochemistry.

Another consideration in the preservation of biological material is the widespread use of pH treatments such as acid shock for probiotics to induce stasis before drying or alkalization of biotech products to cause precipitation for purification. While effective in helping to increase the number of organisms that survive the drying process and remain dormant during storage, residual acids and bases can be problematic when the product is subsequently used or administered.

The modern approach to rectifying this issue is through either 1) decreasing the drying time, or 2) modifying the drying process such that the energy characteristics do not impact the living cells as strongly.

Currently, the processes used to achieve this result often revolve around freezing or freeze-drying. Specifically, the modern approaches include freeze-drying, which involves freezing a product, lowering the pressure and removing the ice through sublimation, or through freezing immediately after blanching, wherein 50%-60% of water is blanched prior to a freezing process.

The present invention seeks to disclose a method of further improving the food preservation process by combining the freezing or freeze-drying process with a partial infrared dehydration step. By using infrared radiation energy to perform simultaneous blanching and dehydration, the drying speed is greatly increased and better cellular and molecular integrity is maintained. Infrared energy may also be targeted to acids used in acid shock treatment to decrease residual acid. This combined with specific freeze-drying or freezing methodologies will result in an improved process that uses less energy, retain better cellular and molecular integrity and greater viability for the preservation of living materials.

### SUMMARY OF THE INVENTION

Dehydrofreezing is a method that is used primarily for the preservation of food and other biological material, wherein the water content of the item is partially removed or minimized to a desirable level through the process of dehydration, followed by, or simultaneously with, freezing the item. This process reduces the potential physical and chemical damage caused by freezing certain items that lack cell structure flexibility, such as fruits, vegetables, nuts, grains and seeds. Similarly, dehydro freeze-drying is a method that combines the process of dehydrofreezing with freeze-drying. Under this method, an airtight vacuum is often utilized during the freezing process for further preservation.

Dehydration is considered to be the most energy consuming step in food and agricultural product processing and preservation, and is typically achieved through the use of hot air, usually generated by gas-fired heaters and electrically driven blowers, directed through an air tunnel or cabinet This process suffers from relatively long drying times, high energy consumption, emitting harmful off-gases such as Nitrogen dioxide, and unpredictable microbial counts in the finished products. Moreover, long drying times can cause significant losses of volatile compounds, reducing the desirable flavor, color and nutritional characteristics of the finished products. Further, high drying temperature and high airflow rate also combine to cause deterioration in the quality of finished products.

Food processing also frequently requires a freezing step. One approach that has been attempted is to combine blanching, dehydrating, and freezing. Such products are often referred to as “dehydrofrozen products.” The dehydrofreezing process offers several advantages over conventional freezing, including (1) energy savings due to lower water load to the freezer; (2) reduced costs related to transport, storage and wrapping; (3) better quality and stability (color, flavor and structure); and (4) superior thawing properties (low drip loss, better color retention and visual characteristics); (5) preservation of molecular and cellular integrity. The moisture content of typical dehydrofrozen products is reduced 40-60% of the original content. These products also need to be processed quickly in order to mitigate the quality change caused by the blanching heat. Conventional convective drying, which is how these products are normally processed, is problematic because it requires a relatively long period which often results in product deterioration.

These two processes are most commonly used in the preservation of many items, including fruits, vegetables, fresh produce, nuts, grains, cannabis, or even other bio organic matter; however, these methods of preservation can also be applied to other biologically active materials such as vaccines and probiotics. The commonality between vaccines, probiotics and products of fermentation or synthetic biology regarding the importance of freeze-drying is that all of them are derived from living systems. When you use thermal energy on that system in order to dehydrate it, the thermal energy can be destructive to biologically active material. For example, in the case of probiotics, that means that using thermal dehydration will result in a decrease in the number of viable bacteria and thus render the probiotic material less effective or even ineffective. In the case of vaccines, a live vaccine is very similar to probiotics. In an inert or dead vaccine, the antigenic properties of proteins that create the necessary immune response for antibody formation are denatured, in the same way that highly specific molecules created by fermentation and synthetic biology can be altered by thermal energy, thus rendering them less effective or even ineffective.

Another issue as to why dehydrofreezing and dehydro freeze-drying are not often used as preservation methods is that they are both extremely cost prohibitive. The expense is primarily based on the amount of time and energy required to dehydrate and freeze an organic or biologically active item so as to not compromise the quality or effectiveness of the item.

The present invention is a system and method for both dehydrofreezing and dehydro freeze-drying that decreases the length of time and costs necessary for the dehydrofreezing and dehydro freeze-drying process to be achieved effectively, while at the same time preserving the viability of the biological material or the integrity of the organic matter. The present invention is able to significantly decrease drying time as well as use a specific thermal energy wavelength that is less likely to break down biological molecules and cell structures, thereby having a minimal impact on living systems and increasing the viability of the preserved content.

In accordance with the present invention, the processes of dehydrofreezing or dehydro freeze-drying are combined with medium to far spectrum infrared (“MFSI) or medium to far spectrum technology, intended to be a replacement for current steam, water and/or microwave blanching and dehydration methods. This invention implements infrared radiation energy for the dehydration of produce or other biologically active material.

MFSI can also can be combined with heated air, microwaves, radio waves, and/or vacuum to accelerate the drying process. Vacuum also enhances heat penetration, thus making the blanching process itself more effective. The vacuum should be in the range of 20-30 inches Hg. The combined infrared and vacuum process also improves the texture and appearance of the finished products.

In general, the advantages of MFSI technology is that it can treat a wide range of products while incorporating: (1) uniform heating which enhances energy efficiency and limits damage from over-heating, (2) capability of zone heating to address differential density, (3) ability to treat large or small lots with the same piece of equipment, (4) portability, since equipment can be built on wheels, (5) the ability to tune wavelengths to deliver energy only to specific types of molecules, and (6) a safe, non-toxic process with no harmful side-effects to humans and reduced environmental impact.

MFSI technology is inherently energy efficient due to the penetration capability of infrared and the elimination of the need for water or steam and can result in significant energy and water savings because infrared penetrates food materials without heating the surrounding air. Another advantage of the invention is that blanching and drying can be achieved in a single step rather than the two steps used at present. This results in simpler processing and saves time.

A major advantage of the invention is that products treated by MFSI technology retain more nutrients, phytochemicals, flavors and physical characteristics compared to blanching conducted with water, steam or microwave energy.

Once the food item or biologically active item, such as probiotic material or vaccines, are dehydrated using MFSI technology, they are then able to be frozen and packed. This process is the dehydrofreezing process, whereas the dehydro freeze-drying process involves the incorporation of vacuum methodology after the item is dehydrated and frozen.

### BRIEF DESCRIPTIONS OF THE DRAWINGS

FIG. 1 is a flow diagram of the dehydrofreezing process according to one embodiment of the present invention;

FIG. 2 is a flow diagram of the dehydro freeze-drying process according to one embodiment of the present invention.

### DETAILED DESCRIPTION OF THE PREFERRED EMBODIMENT

As a preliminary matter, as used herein, the term “food products” or “product” refers to any fruits, vegetables, seeds, nuts grains, fungi, tuber and cannabis, and other biological compounds including but not limited to: viruses, bacteria, probiotics, fermentates, metabolites, synthetic bio organic matter, and pharmaceuticals and each of them.

The process of preserving food products includes a process known as blanching. Blanching is a cooking process wherein a food is exposed to either saturated steam or hot water. This step is done prior to drying the item. The present invention seeks to supplement that process using medium to far field infrared radiation (IR) to perform the drying or blanching process. IR effectively transfers energy and penetrates food products, thereby effectively removing naturally present moisture. In addition, unlike traditional steaming the IR process is known to preserve or disable particular enzymes that are present in various food products. The present invention further couples the IR blanching process with novel freezing or freeze-drying techniques. This combination has been previously unknown.

While some traditional methods of IR rely on the blanching process alone, the present invention further seeks to improve the preservation process immediately taking the blanched food item and subjecting it to a freezing process. By freezing the blanched food, the preservation process is more efficient and allows for the food to rehydrated more quickly and last longer.

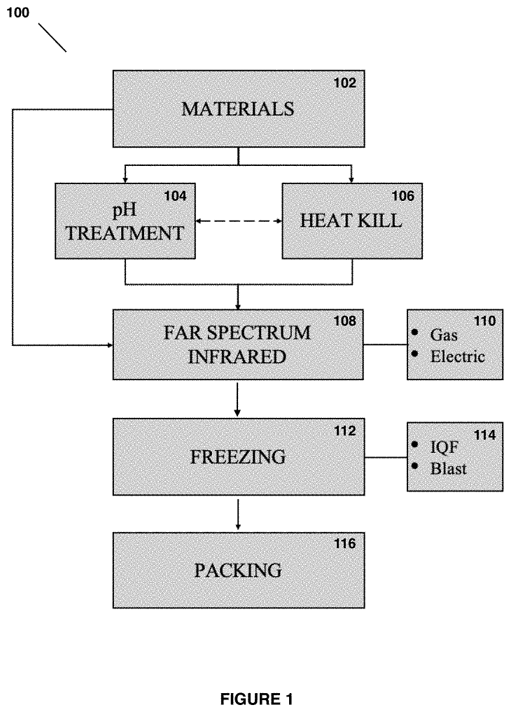

FIG. 1 is a flow diagram of one embodiment of the dehydrofreezing 100 aspect of the present invention. In accordance with the preferred embodiment of the present invention, materials 102 or food products are preserved through either a pH treatment 104 or with heat 106 to remove harmful pathogens, followed by processing using far spectrum infrared radiation 108, which may be powered by either gas or electric means 110. The processed materials undergo a freezing process 112, which may be done using Individual Quick Freezing (“IQF”) or blast freezing methods 114. The frozen processed material is then packed 116 and ready to be stored or distributed.

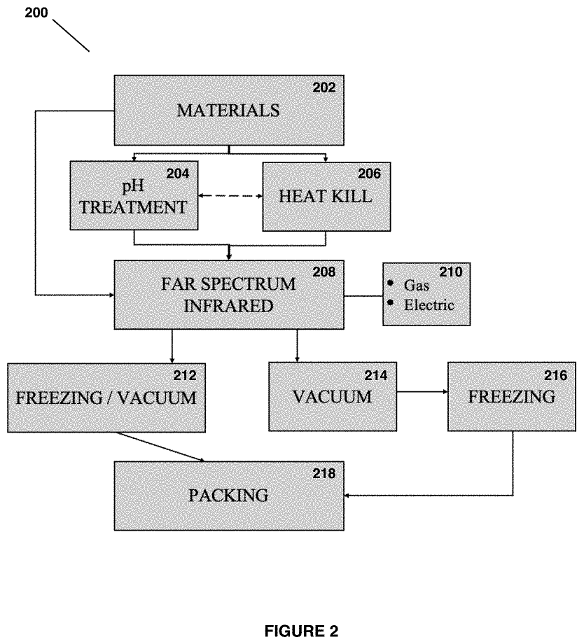

FIG. 2 is a flow diagram of the dehydro freeze-drying process 200. In accordance with the preferred embodiment of the present invention, materials 202 such as food products are preserved through either a pH treatment 204 or with heat 206 to remove harmful pathogens, followed by processing using far spectrum infrared radiation 208, which may be powered by either gas, photonic, electric or equivalent method 210. The processed materials undergo a combined freezing/vacuum process 212, or can vacuum sealed 214 and then frozen 216. In other embodiments, the items are first frozen, then vacuum sealed. The frozen, vacuum packed and processed material is then packed 218 and ready to be stored or distributed for immediate use.

Recently, new and improved infrared heaters or emitters with appropriate wavelengths have been developed, which makes the application of the technology to food and agricultural pharmaceutical, or other biological processing possible. The new and improved heaters or emitters with appropriate wavelengths provide much more control, permitting more specific and precise treatment of food and agricultural products. Infrared radiation energy can be generated by converting thermal or electric energy to infrared radiation energy. Various infrared emitters have been developed: catalytic, electric, carbon, laser, gas and ceramic. IR emitters work by transferring a large amount of thermal energy to both the surface and interior of the food products being processed.

This radiation energy heats the product to a target temperature in order to achieve blanching and drying simultaneously. Infrared radiation itself is energy in the form of a band of invisible light or electromagnetic wave. Depending on specific wavelength range, infrared energy generally is divided into the following categories: near infrared (0.8-2 um), medium infrared (2-4 um) and far infrared (4-100 um). Molecular (chemical) bonds, present in all Substances, evince certain physical phenomena such as vibrational and rotational frequency. IR radiation is able to excite or increase the vibrational or rotational frequency of these bonds, thereby generating heat in the product being treated.

In one embodiment of the present invention, the IR transmitter is an IR laser transmitter. A laser is used to remove water and liquids from a fruit, vegetable, or the like. Traditional steam drying is typically facilitated by macerating the outer skin of the produce (often by knife blade, pin, needle, or the like), to more readily allow the water to be removed. This process can damage the food item. However, radiation by a laser overcomes this drawback by introducing one or more holes that penetrate through the outer, protective layer to allow the moisture to be rapidly evacuated from the target. These holes are large enough to allow water molecules to escape and multiple, uniformly spaced holes allow for faster and more uniform processing of the target. This process generally works with any type of frozen, freeze-dried and/or dehydrated vegetables or fruits, such as carrots, corn, beans, peas, apples, peaches, plums, pears, cherries, cranberries, or the like, to give but a few examples.

In one embodiment, the blanching process is done through electron beam irradiation. Electron-beam irradiation (EBI) is a novel, non-thermal, physical method of food preservation (processing) technology which is effective in achieving microbial decontamination, insect disinfestation and shelf-life improvement of various food products. This technology is economical and environmentally friendly and holds several advantages over other sources of food irradiation and conventional preservation techniques. Based on the available scientific reports, EBI could prove to be a potential alternative to the current chemical fumigants used for preservation purposes. Reports available have clearly indicated the effectiveness of employing electron beams in preserving the overall qualities and extending the shelf life of various fruits, vegetables, cereals, legumes, poultry, meat and seafoods. EBI can be highly effective when combined with other conventional and non-conventional food-processing technologies.

Another method of food preservation relates to the use of cold plasma. Cold plasma refers to a state of matter involving a collection of free-moving electrons and ions. Usually, high energy is needed to produce it; such as a sudden electrical discharge (lightning) or nuclear fusion (a star). Thus, it is generally tricky to make plasmas at atmospheric pressures and room temperatures. The term “cold plasma” or “cold atmospheric plasma” as used herein refers to a recently discovered process by which electrons are superheated to thousands of degrees. Shortly thereafter, a few of the molecules are ionized in the plasma, allowing the heat to be distributed to non-ionized molecules, making it cool—or at least lukewarm—enough to handle. Several different gases can be used to produce Cold Atmospheric Plasma such as Helium, Argon, Nitrogen, Heliox, and air.

Cold plasma can offer numerous benefits over present methodologies related to freeze drying, since blasts of cold plasma have been shown to kill drug resistant bacteria in food products. For example, recent studies have shown that cold plasma blasts, or treatment, can result in a 99.9% reduction of norovirus in blueberries, without damaging the fruit. Scientists have further been able to use cold plasma to kill pathogens such as salmonella, E. Coli on various fruits.

One embodiment of the present invention seeks to treat food products with a cold plasma (which may be enabled at any desired temperature including at ambient temperature), thereby reducing harmful bacteria without affecting the food product in a negative manner.

In another embodiment, laser treatment of food products enhances, or enables, creation of nutritionally enhanced or fortified foods and/or the possibility of functional and nutraceutical foods. For example, some foods lose their nutritional value at various steps during the processing chain due to the nature of the processing. Alternatively, various additives may be put into certain foods to enhance their functionality, nutritional provision, or even medicinal effectiveness.

One embodiment of the present invention utilizes pulsed electric field treatment to kill vegetative bacteria and yeasts. Pulsed Electric Field (PEF) treatment is defined as the application of short burst of high intensity electric field pulses in the range of 20-80 kV/cm for very short treatment time of micro to milliseconds to pasteurize foods. PEF processing usually applied at ambient or little under or above ambient temperature and in addition to short processing time, heat generation during PEF process is minimized, and process remains non-thermal. After it is first practiced in 1930s, now it is one of the most studied non-thermal emerging technologies to process low viscosity high acidity food products especially fruit and vegetable juices, milk with low amount of fat, soups, and sauces.

PEF processing as a function of electric field strength, electrical energy and treatment time does not cause detrimental changes on physical, biochemical and sensory properties of food samples as well as bioactive compounds. Moreover, PEF processing provides inactivation of spoilage and foodborne pathogens as well as enzymes that cause microbial spoilage and downgrading quality, respectively. Studies with microbial reduction involve inactivation of Escherichia coli, Escherichia coli O157:H7, Salmonella sp., Listeria monocytogenes, Listeria innocua, Bacillus cereus, Pseudomonas fluorescence and Saccharomyces cerevisiae, etc., whereas enzyme inactivation studies include inhibition of Pectin Methyl Esterase (PME), lipoxygenase, polygalacturonase, Peroxidase (POD), Polyphenoloxidase (PPO), and β-glucosidase.

Killing of vegetative bacteria and yeasts by PEF processing is likely not due to the products of electrolysis or temperature increase alone, but rather by the applied electrical field strength and the processing time. There are a few theories on the mechanisms involved in the disruption of the cell membrane when subjected to electric fields. Two hypotheses, electrical breakdown and osmotic disproportion, are widely approved and are supported on the same principles. The theory of electrical breakdown considers the cell membrane as a condenser loaded with a dielectric medium. Accumulation of free charges at the internal and outer surface of the cell membrane forms a Transmembrane Potential (TMP) of approximately 10 mV. When an external electrical field is applied; ions inside and outside of the cell move along with the electric field until they are restrained and accumulated at the membrane causing a rise in the TMP. The ions of opposite charge (+ and —) on each side of the membrane are pulled to each other, squeeze the membrane and cause a decrease in its thickness. Further application of electric fields causes more stress on cell membrane and reduction in its thickness ends up with pore formation when applied electric field strength is above the critical electric field potential of the cell membrane. If the application of electric field continues, the pores become irreversible and cells cannot reseal themselves leaking of intracellular materials, and thus, cell death occurs. The principle of osmotic irrationality, on the other hand, defines the imbalance of cell membrane components through the formation of hydrophilic pores in the membrane and the opening of the protein channels. Applied electric field causes structural changes in the conformation of phospholipids, ending up the rearrangement of the membrane structure and constitution of hydrophilic pores.

In any event, using a laser to put holes into various foods enhances or enables the freeze-drying process to occur more effectively than the current methodologies. In this method, the wavelength of the laser beam thas a focused laser spot. The method comprises the step of applying a laser pulse with a pulse duration in the range of 1˜1000 fs the food material. In the step, convergence laser spot is located on or in the food material in the body surface of the food material, laser pulse creates a cavity in the food material at the position of the focused laser spot. The term “near-infrared (IR) range” refers to the wavelength range of about 750˜1400 nm. The term “cavity” is dependent on the position of the focused laser spot, pointing a hollow space or recess that is formed on the surface or inside of the food material. Region cavity is formed essentially because it is limited to the position of the focused laser spot, the size of the cavity created by the laser pulse is substantially determined by the size of the laser spot. In conventional optical techniques, several microns ([mu] m) or 1 micron ([mu] m) the size of the well of the laser spot even less can be easily achieved. As a result, the cavity formation can be restricted to a very small area or volume. Therefore, reducing the pulse duration leads to an additional increase in the accuracy with which the cavity can be formed in the food products. It is essentially an advantage when the processed food material is extremely susceptible to thermal damage. The energy is not generated substantially thermally outside the position of the laser spot. Thus, cavity, with a high degree of precision, and, with respect to the material surrounding the cavity, the food material is created without significant damage.

In one embodiment, the method of the present invention comprises the step of applying a laser pulse to successive having a pulse duration in the range of 1˜1000 fs to the food material, in said step, the focused laser spot, lies on the surface of the food material or in the food material, which creates a cavity in the food material at the position of the focused laser spot.

Pulse duration is preferably in the range of 1˜800 Fs, and more preferably within a range of 1˜400 Fs. When duration of the applied laser pulse or pulses is short, the amount of energy accumulated in the food material per laser pulse is small. Therefore, a decrease in pulse duration yields a further increase in the precision with which the cavity is formed in the food material.

This is particularly beneficial for the case where the food material extremely sensitive to thermal damage is processed. Repetition rate of successive laser pulses is preferably in the range of 1˜1000 MHz. Number of repetitions of this order, in particular, when used in combination with a high-speed laser scanner or positioning device, allows for fast processing of food materials.

Another method of infrared blanching is through a flameless gas-fired infrared radiation emitter. It is important to note that, in general, IR equipment can be designed and operated in two different heating modes, continuous or intermittent heating. During continuous heating, the radiation intensity is maintained constant by retaining a continuous or intentionally varied supply of gas to the emitter. Keeping a product temperature constant can be achieved through intermittent heating, which is normally achieved by using natural gas or electricity . A variation of this method is to use an interval method of alternating between heating and cooling, in declining times for the heat, as more and more moisture is driven off. This results in the water evaporating, but not the volatiles, when there is enough moisture in the substance. The present invention discloses a method of using an infrared gas emitter in the blanching process. The appropriate heating mode and conditions are determined based on the application and the property of the materials. For quick heating or enzyme activation or deactivation, continuous heating is advantageous since it delivers a constant high energy to the surface or within the food products. For certain fruits and vegetables, continuous heating may be beneficial to remove moisture. However, often times continuous heating in food products has been known to cause surface discoloration. Intermittent heating is best for maintaining volatiles, allowing for increased IR and resulting in more moisture being evaporated.

In another embodiment, the drying is done after blanching. In those situations, intermittent heating may work best in the drying stage, since it tends not to cause severe surface darkening by regulating the product temperature. Intermittent heating also provides benefits in terms of energy usage.

Probiotics are living microorganisms which upon consumption in adequate quantities via ingestion confer beneficial effect on health beyond inherent basic nutrition. Lactic acid bacteria and bifidobacteria are between the most common microorganisms used as probiotics. Mechanisms such as immunomodulation, growth inhibition of pathogens in the gastrointestinal and urogenital tract and improved intrinsic defensive mechanisms, which may be through production of hydrogen peroxide, organic acids, bacteriocins and the release of biosurfactants, are involved in the probiotic effect.

Because of their generally accepted benefits, probiotics during recent years have gained wide interest and represent an alternative to previous therapies. Freeze-drying is a commonly used technique for the production of dried powders of probiotics. In this process, probiotics are exposed to damage from the process conditions such as very low freezing temperatures and dehydration. Cells are first frozen to below the critical temperature of the formulation, and then dried by sublimation under high vacuum in two phases: primary drying, during which unbound water is removed and secondary drying, during which the bound water is removed. These stages can damage the constituents of the cell wall and lead to cell death. However, the presence of cryoprotectants in the drying medium increases the viability of cells after drying (8). It is important to optimize the production process of probiotic preparations in order to obtain a product with suitable properties and higher number of viable probiotic microorganisms. Among several probiotic preparations, there has been an increasing interest in the development of dried formulations.

Moreover, a variety of cryoprotectants have been used for lyophilization (another name for freeze-drying) of probiotics in order to increase the survival rate of microorganisms after freeze-drying. The role of cryoprotectants, such as skim milk powder, whey protein, trehalose, glycerol, betaine, adonitol, sucrose, glucose, lactose and polymers, have been investigated. It is technologically and economically reasonable to assess the influence of these compounds on the survival rate of probiotic bacteria and to verify a suitable combination which provides an effective medium for lyophilization. However, limited data are available on the effect of various combinations of cryoprotectants on the stability of Lactobacillus strains during freeze-drying process. Furthermore, most studies have evaluated the survival of lactobacilli only during the freeze-drying process and not during the storage of dosage forms.

An issue seen with many freeze—dried probiotic formulations is when they are added to liquid preparations prior to storage (seen in many yogurts and other food-based products), the result is that ultimately rehydrating the freeze—dried bacteria decreases the stability of the probiotics affecting their viability through storage This issue was investigated by Weinbreck et al. which showed that dried encapsulated Lactobacillus bacteria when exposed to water over a 2-week period significantly decreased the viability of the encapsulated bacteria further proving that even when encapsulated bacteria will have to remain dry to the point of delivery to have the viability needed to exert the required health benefits. This decrease was further explained by Vesterlund et al. who showed that when dried foods containing probiotic bacteria was exposed to or contained water, the viability of probiotic bacteria during the shelf-life of the product decreased considerably. Over-drying of probiotic bacteria however can be detrimental in the bacterial survival rate over time. This was due to the biological nature of bacterial cells, where a 0.0% moisture content revealed a bacterial viability decrease of 44% within 1 week of storage when compared to bacteria containing a moisture content of 2.8%. It was therefore determined that an ideal moisture content for the probiotic bacteria analyzed, Lactobacillus salivarius, was between 2.8% to 5.6% where a moisture content of 8.8% and over resulted in a large decrease in bacterial viability over time. These values were however specific to the bacteria tested and would vary from species to species.

An alternative to cryo-protection is the use of microencapsulation, which is also used to protect probiotic bacteria during freeze-drying. Microencapsulation using polysaccharide or protein-based systems has been shown to be far more effective in the protection of bacteria during freeze-drying and storage as compared to traditional cryo-protection. Combined with the effect of polysaccharides, some of which are used as prebiotics, this allows for a suitable delivery system that protects the delivered probiotic bacteria and has the added effect of producing a synbiotic formulation. Prebiotics by definition, provide growth enhancers and nutrients that assist in the growth of probiotic bacteria when delivered to the small intestine. Synbiotics are defined as a “combination of pre- and probiotics.” The most commonly used prebiotics in Europe are fructo-oligosaccharides [FOS], which are naturally found in a variety of vegetables such as asparagus, leeks, artichokes, onions, and garlic.

The parameters of the freeze—drying process have also been shown to have a large effect on bacterial viability. This effect has been shown to be strain specific with certain species of probiotic bacteria being capable of surviving lower temperatures when compared to other bacterial species. An example of a bacterium that is unstable at low temperatures is Lactobacillus delbrueckii, a probiotic whose numbers decrease drastically at temperatures below 0° C. In comparison, L. paracasei, has been shown to survive at much lower temperatures, commonly associated with freeze-drying, with a significantly larger proportion of bacterial cells surviving the formulation process. This difference was shown to be attributed to the membrane structure of the respective bacterial cells affecting the resistance of the bacteria against low temperatures. Low temperature vacuum drying (LTVD) is therefore proposed as an alternative to freeze-drying due to the lower temperature ranges utilized and higher viable bacteria yields seen in cryo-labile bacteria such as L. delbrueckii.

Storage conditions of probiotics before and after formulation processes have also been shown to be an important factor in the viability of the delivered probiotic bacteria. Probiotics have been shown to survive in greater numbers when stored at −70° C. prior to the formulation process compared to when stored at 7° C. in a refrigerator. This was due to the cryo-protectants used such as glycerol, milk, etc. that prevented intracellular formation of ice within the bacteria, thus preventing a decrease in their viability when frozen. The issue that arises, however, from storing probiotic bacteria at frozen temperatures is the problem of transportation and cold storage across great distances. This can be solved by transportation of cultures to the site of culturing and processing and maintaining a cold chain from production to delivery It was further shown that the presence of other bacteria in the formulation, oxygen content, the amount of acid-producing bacteria as well as the temperature affected the viability of probiotic bacteria in liquid or semi-solid food based product such as yogurts.

Food processing often requires the freezing step. Attempts have been made to combine a method of blanching, freezing and dehydration. These products are usually referred to as “frozen dehydrated products.”

Compared with the conventional freezing method, freezing of dehydrated food products provide certain advantages, including (1) efficiency due to the low water chiller energy savings; (2) reduce the costs associated with the transportation, storage and packaging; (3) better quality and stability (color, flavor and structure); and (4) excellent melting properties (low loss washout). Typical moisture content of the frozen product is dehydrated to reduce the initial content of 40-60%. These products also need to be processed rapidly in order to reduce the blanching mass change due to heating, which often leads to deterioration of the product.

Generally, high food quality with low dehydration losses is obtained using low temperature liquid nitrogen freezing systems which operate at about −320° F. Ammonia and freon vapor compression mechanical systems, which operate at relatively high temperatures, such as −40° F., are commonly used to freeze food in an economical manner, but with high freezing times and high dehydration losses. Recently, high performance vapor compression, mechanical systems have emerged which produce high quality frozen foods with low dehydration losses at relatively high temperatures of from −40° F. to −60° F. Because they operate at such relatively high temperatures, dehydration losses associated with high performance mechanical freezers leave room for improvement. They typically also cannot operate at low temperatures due to limitations associated with common refrigerants. If a low temperature refrigerant system could be developed, dehydration losses can be appreciably reduced.

Some novel food freezing technologies, including high-pressure freezing (HPF), ultrasound-assisted freezing (UAF), electrically disturbed freezing (EF) and magnetically disturbed freezing (MF), microwave-assisted freezing (MWF), and osmo-dehydro-freezing (ODF). HPF and UAF can initiate ice nucleation rapidly, leading to uniform distribution of ice crystals and the control of their size and shape. Specifically, the former is focused on increasing the degree of supercooling, whereas the latter aims to decrease it. Direct current electric freezing (DC-EF) and alternating current electric freezing (AC-EF) exhibit different effects on ice nucleation. DC-EF can promote ice nucleation and AC-EF has the opposite effect. Furthermore, ODF has been successfully used for freezing various vegetables and fruit. MWF cannot control the nucleation temperature, but can decrease supercooling degree, thus decreasing the size of ice crystals. The heat and mass transfer processes during ODF have been investigated experimentally and modeled mathematically.

Freeze-drying is the removal of ice or other frozen solvents from a material through the process of sublimation and the removal of bound water molecules through the process of desorption.

Lyophilization and freeze-drying are terms that are used interchangeably depending on the industry and location where the drying is taking place. Controlled freeze-drying keeps the product temperature low enough during the process to avoid changes in the dried product appearance and characteristics. It is an excellent method for preserving a wide variety of heat-sensitive materials such as proteins, microbes, pharmaceuticals, tissues & plasma.

In freeze-drying, two basic freezing methods are in use; namely, prefreezing and evaporation freezing. In prefreezing, the material is first frozen by refrigeration equipment before being placed in a vacuum chamber for sublimation, whereas in evaporation-freezing the material is placed in the unfrozen state in the chamber, and freezing is carried out by the cooling action which accompanies evaporation.

Freeze-drying is easiest to accomplish using large ice crystals, which can be produced by slow freezing or annealing. However, with biological materials, when crystals are too large they may break the cell walls, and that leads to less-than-ideal freeze-drying results. To prevent this, the freezing is done rapidly. For materials that tend to precipitate, annealing can be used. This process involves fast freezing, then raising the product temperature to allow the crystals to grow.

Freeze-drying's second phase is primary drying (sublimation), in which the pressure is lowered and heat is added to the material in order for the water to sublimate. The vacuum speeds sublimation. The cold condenser provides a surface for the water vapor to adhere and solidify. The condenser also protects the vacuum pump from the water vapor. About 95% of the water in the material is removed in this phase. Primary drying can be a slow process. Too much heat can alter the structure of the material.

Freeze-drying's final phase is secondary drying (adsorption), during which the ionically-bound water molecules are removed. By raising the temperature higher than in the primary drying phase, the bonds are broken between the material and the water molecules. Freeze dried materials retain a porous structure. After the freeze-drying process is complete, the vacuum can be broken with an inert gas before the material is sealed. Most materials can be dried to 1-5% residual moisture.

In one embodiment, the refrigeration system cools the (ice) condenser located inside the freeze dryer. The refrigeration system can also be employed to cool shelves in the product chamber for the freezing of the product. The vacuum system consists of a separate vacuum pump connected to an airtight condenser and attached product chamber.

Control systems vary in complexity and usually include temperature and pressure sensing ability. Advanced controllers will allow the programming of a complete “recipe” for freeze-drying and will include options to monitor how the freeze-drying process is progressing. Choosing a control system for the freeze dryer depends on the application and use (i.e. lab vs. production).

Product chambers are typically either a manifold with attached flasks, or, a larger chamber with a system of shelves on which to place the product. The purpose of the condenser is to attract the vapors being sublimed off of the product. Because the condenser is maintained at a lower energy level relative to the product ice, the vapors condense and turn back into solid form (ice) in the condenser. The sublimated ice accumulates in the condenser and is manually removed at the end of the freeze-drying cycle (defrost step). The condenser temperature required is dictated by the freezing point and collapse temperature of the product. The refrigeration system must be able to maintain the temperature of the condenser substantially below the temperature of the product.

In shelf freeze dryers, the condenser can be located inside the product chamber (internal condenser) or in a separate chamber (external condenser) connected to the product chamber by a vapor port. Manifold freeze dryers rely on ambient conditions to provide the heat of sublimation to the product. This heat input does not melt the product because an equivalent amount of heat is removed by vaporization of the solvent. Advanced shelf freeze dryers can provide a heat source to control/expedite the drying process and they can also employ the refrigeration system to allow freezing of product inside the unit.

Freeze dryers can be informally classified by the type of product chamber: (1) Manifold dryers where the product is typically pre-frozen & in flasks (2) Shelf dryers where the product is placed in a tray or directly on a shelf (3) Combination units with both drying options.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

What is claimed is:

1. A method of preserving biological material comprising blanching the biological material using partial infrared dehydration prior to freezing the biological material.

2. A method of preserving or producing biological material, the method comprising:
sufficiently reducing the moisture content of the biological material by exposing it to medium to far field infrared radiation;
the medium to far field infrared radiation produced by an infrared emitter; and
subsequently reducing the temperature of the biological material for long term preservation.
